(19) United States
(12) Patent Application Publication
(10) Pub. No.: US 2013/0085380 A1
(43) Pub. Date: Apr. 4, 2013

(54) SYSTEMS AND METHODS FOR PLANNING IMAGE GUIDED INTERVENTIONAL PROCEDURES

(71) Applicant: Gnanasekar Velusamy, Tamilnadu (IN)

(72) Inventor: Gnanasekar Velusamy, Tamilnadu (IN)

(73) Assignee: PERFINT HEALTHCARE PRIVATE LIMITED, T'Nagar (IN)

(21) Appl. No.: 13/435,980

(22) Filed: Mar. 30, 2012

Related U.S. Application Data
(63) Continuation of application No. 13/292,327, filed on Nov. 9, 2011.

(30) Foreign Application Priority Data
Nov. 10, 2010 (IN) 3344/CHE/2010
Nov. 10, 2010 (IN) 3348/CHE/2010
Aug. 19, 2011 (IN) 3344/CHE2010
Aug. 19, 2011 (IN) 3348CHE/2010

(51) Int. Cl. A61B 6/03; A61B 5/055; A61B 18/04; A61B 6/00; A61B 18/18; A61B 18/02; A61B 5/06; A61B 5/00

(52) U.S. Cl. CPC A61B 6/032; A61B 5/06; A61B 5/055; A61B 5/742; A61B 6/52; A61B 18/1815; A61B 18/18; A61B 18/02; A61B 18/04
USPC 600/424

(57) ABSTRACT

In some embodiments, a planning station can receive image data associated with an image(s) of an area of interest within a body of a patient and display the image(s) on a display device. A user can make a selection of a first interventional tool and a second interventional tool about which information is stored in a memory of the planning station. The planning station can execute a simulation viewable on the display device of a treatment plan for disposing the first and second interventional tools in the body of the patient and applying thermal energy from the first and second interventional tools to the body of the patient. The planning station can generate a thermal model of the thermal effect collectively produced on tissue of the patient by the first interventional tool and the second interventional tools and display the thermal model on the display device.

### CROSS-REFERENCE TO RELATED APPLICATIONS

This application is a continuation application of U.S. patent application Ser. No. 13/292,327, entitled “Systems and Methods for Planning Image-Guided Interventional Procedures,” filed Nov. 9, 2011, which claims priority to and the benefit of Indian Non-provisional Patent Application No. 3348/CHE/2010, entitled “Planning Station,” filed Aug. 19, 2011, which claims the benefit of Indian Provisional Patent Application No. 3348/CHE/2010, entitled “Planning Station,” filed Nov. 10, 2010, and Indian Non-provisional Patent Application No. 3344/CHE/2010, entitled “System for Performing Ablation Procedures,” filed Aug. 19, 2011, which claims the benefit of Indian Provisional Patent Application No. 3344/CHE/2010, entitled “System for Performing Ablation Procedures,” filed Nov. 10, 2010, the disclosures of which are hereby incorporated by reference in their entirety.

### BACKGROUND

The invention relates to systems and methods for image-guided procedures, and more particularly to systems and methods for the planning and execution of image-guided interventional procedures.

Some known interventional procedures include the manual insertion of an interventional tool, which can be prone to the risk of damaging neighboring tissues or organs. In some known interventional procedures, to limit or prevent such potential damage, the interventionist performs the procedure very cautiously, which can make the procedure very time consuming. In some known image-guided interventional procedures, manual insertion of an interventional tool may not be precise as the imaging is not performed in real time and the position of the tool may not be visible due to the minimally invasive nature of the procedure. Such procedures can also be time consuming as the interventionist may have to move the interventional tool by very small increments between control scans to ascertain the position of the tool.

Thus, a need exists for a system and method of planning an image-guided interventional procedure that can allow be used to assist the interventionist in the accurate placement of one or more interventional tools to treat a target tissue (e.g., a tumor) to avoid collisions with surrounding tissues and organs when multiple interventional tools are used.

### SUMMARY OF THE INVENTION

Systems and methods for use in an image-guided interventional procedure are described herein. In some embodiments, a planning station can receive image data associated with an image(s) of an area of interest within a body of a patient and display the image(s) on a display device. A user can make a selection of first interventional tool and a second interventional tool about which information is stored in a memory of the planning station. The planning station can execute a simulation viewable on the display device of a treatment plan for disposing the first and second interventional tools in the body of the patient and applying energy from the first and second interventional tools to the body of the patient. The planning station can generate a thermal model of the thermal effect collectively produced on tissue of the patient by the first interventional tool and the second interventional tools and display the thermal model on the display device.

### BRIEF DESCRIPTION OF THE DRAWINGS

FIG. 1 is a schematic illustration of a system for use in an image-guided interventional procedure, according to an embodiment.

FIG. 2A is a schematic illustration of a planning station, according to an embodiment.

FIG. 2B is an illustration of a planning station, imaging device and robotic positioning device according to an embodiment.

FIG. 3 is a schematic illustration of a display showing images produced during a procedure to target a tissue to be treated.

FIG. 4 is an illustration of a tissue block generated by a thermal ablation module showing an ablation tool disposed at a center of the tissue block.

FIG. 5 is an illustration of an example model of ablation regions generated by the thermal ablation module.

FIG. 6A is an example of a surface plot of an ablation tool with a 30 mm radiating element and power applied at 100 W for a duration of 600 seconds, generated by the thermal ablation module.

FIG. 6B is an example of a surface plot of three ablation tools each having a 40 mm radiating element and power applied at 200 W for a duration of 600 seconds, generated by the thermal ablation module.

FIG. 7 illustrates an estimation of an iso-surface point during an interpolation method performed by an ablation volume data module.

FIG. 8 is a surface plot illustrating an example estimate of a point on an iso-surface generated by the ablation volume data module.

FIG. 9 is a flow chart illustrating a method for performing a set-up procedure to prepare a positioning device and patient for an image-guided interventional procedure, according to an embodiment.

FIG. 10 is a flow chart illustrating a method for performing a procedure to prepare an imaging device and a patient for images to be taken, and imaging a region of interest on the patient, according to an embodiment.

FIGS. 11-14 each illustrate a portion of a method of generating a plan for an image-guided interventional procedure and a simulation of such plan, according to an embodiment.

FIGS. 15-18 each illustrate a portion of a method of performing an image-guided interventional procedure, according to an embodiment.

FIG. 19 is a schematic illustration of a display showing a 3D image and corresponding 2D slice generated by the planning station, according to an embodiment.

FIG. 20 is a flow chart illustrating a method of performing an image guided interventional procedure, according to an embodiment.

### DETAILED DESCRIPTION

Systems and methods for planning and executing an image-guided interventional medical procedure are described herein. A system as described herein can include a planning station that can be used in conjunction with a robotic positioning device. In some embodiments, the planning station can be configured to plan and place one or more interventional tools, such as, for example, one or more ablation needles, and to determine a collision avoidance path for each tool. An optimal tool positioning sequence can be determined and a path for each tool positioning can be determined. For example, a path of insertion, insertion depth and order of insertion can be determined for each tool. Thus, the planning station can be configured to determine a path of insertion of multiple tools such that the tools do not collide or interfere with each other during an interventional procedure, such as, for example, an interventional ablation procedure.

In some embodiments, the planning station can provide a simulation of the placement, insertion and activation of one or more interventional tools. For example, based on image data of a target treatment area of a patient, the planning station can enable the clinician to select one or more interventional tools to perform the interventional procedure and determine an insertion path and depth for each of the selected interventional tools and can then provide to the physician a virtual simulation of the treatment plan. In some embodiments, for example, where the interventional tools are ablation needles, a thermal model of the target tissue including the collective thermal effects on tissue of the simulated procedure can be provided. Thus, the clinician (e.g., physician or other person performing the procedure) can determine if the selected interventional tools are sufficient to perform the desired treatment. If the thermal model indicates that the target tissue has not been treated to a desired outcome, the simulation can be adjusted or changed before and/or during an interventional procedure.

In some embodiments, a non-transitory processor-readable medium storing code representing instructions to cause a processor to perform a process includes code to receive image data associated with at least one image of an area of interest within a body of a patient and to display the at least one image on a display device. The area of interest includes a target tissue to be treated. The processor-readable medium further includes code to receive an input from a user of a selection of a first interventional tool and a selection of a second interventional tool each from multiple interventional tools about which information is stored in the memory. The processor-readable medium further includes code to execute a simulation viewable on the display device of a treatment plan that includes disposing the first interventional tool in a first location and the second interventional tool in a second location in the body of the patient, applying a first amount of energy from the first interventional tool and applying a second amount of energy from the second interventional tool to the body of the patient. The processor-readable medium further includes code to generate a thermal model of the thermal effect collectively produced on tissue of the patient, including the target tissue, by the first interventional tool and the second interventional tool based on the simulation, and to display the thermal model on the display device.

In some embodiments, a method includes viewing on a display device an image associated with an area of interest within a body of a patient. A selection of a target tissue within the area of interest to be treated based on the image is entered at a planning station. A selection of a first interventional tool and a selection of a second interventional tool to use during an interventional procedure to treat the target tissue is entered at the planning station. Generation of a visualization of a first plan of treatment of the target tissue based on the selecting a target tissue and the selecting a first interventional tool and a second interventional tool is requested from the planning station. During an interventional procedure to treat the target tissue based on the first plan of treatment including insertion of the first interventional tool in the body of the patient, a visualization of a second plan of treatment be generated is requested at the planning station. The second plan of treatment includes a change to at least one of a selected power level of the second interventional tool, a selected time duration of applying the power level of the second interventional tool, or a position of the second interventional tool.

In some embodiments, a non-transitory processor-readable medium storing code representing instructions to cause a processor to perform a process includes code to generate a first plan of treatment for an image-guided interventional procedure including treatment of a target tissue within an area of interest within a body of a patient using a first interventional tool and a second interventional tool. The first plan includes a predetermined first power level and a predetermined first duration of time to apply the first power level for the first interventional tool and a predetermined second power level and a predetermined second duration of time to apply the second power level for the second interventional tool. The processor-readable medium further includes code to, during an interventional procedure executing the first plan of treatment, receive an image signal associated with at least one image of the area of interest within the body of the patient with the first interventional tool inserted therein, and receive from a user executing the first plan of treatment, an input indicating a request to generate a second plan of treatment. The second plan of treatment includes a third power level and/or a third duration of time applying the second or third power level for the second interventional tool.

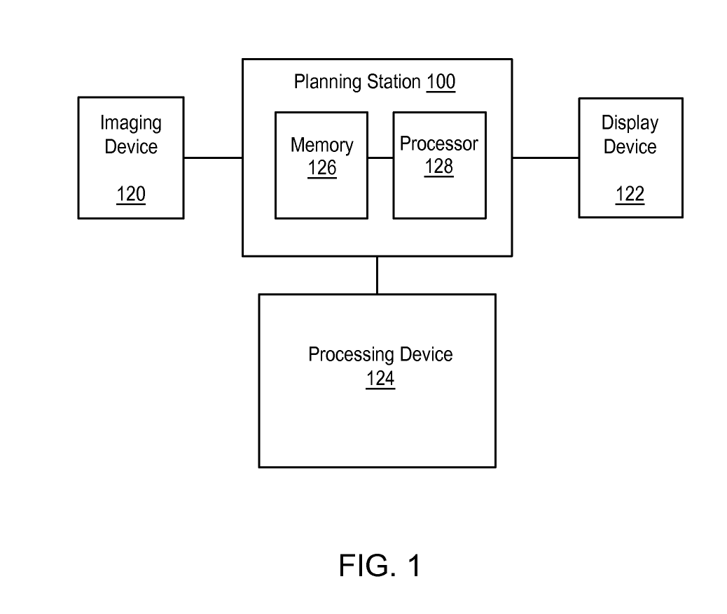

FIG. 1 is a schematic illustration of a planning station 100, an imaging device 120, a display device 122 and a positioning device 124, according to an embodiment. The planning station 100 can be used in conjunction with the imaging device 120 and positioning device 124 to generate a plan for an image-guided interventional procedure and provide a virtual simulation of the plan viewable on the display device 122. In some embodiments, the planning station 100 can be used in conjunction with the imaging device 120 to generate a plan for treatment.

The planning station 100 (also referred to herein as “planning system”) can be an electronic computing device, such as, for example, a personal computer, a laptop computer, a personal digital assistant (PDA), a portable/mobile internet device and/or some other electronic computing device. The planning station 100 can also include or be operatively coupled to a display device 122, a keyboard (not shown), various ports (e.g., a USB port), and other user interface features, such as, for example, touch screen controls, audio components, and/or video components. The planning station 100 can be operatively coupled to a communications network, such as for example, the Internet and include a web browser configured to access a webpage or website hosted on or accessible via a network, such as the Internet. The planning station 100 can include an operating system, such as, for example, Windows XP or Linux.

In some embodiments, the planning station 100, the display device 122 and/or the positioning device 124 are combined into a single device or component. In some, the planning station 100 can include a display device/screen. In some embodiments, the positioning device 124 can include a display device/screen.

The planning station 100 can include one or more processors 128 and one or more memory components 126. The processor(s) 128 can be any of a variety of processors. Such processors can be implemented, for example, as hardware modules such as embedded microprocessors, microprocessors as part of a computer system, Application-Specific Integrated Circuits (“ASICs”), and Programmable Logic Devices (“PLDs”). Some such processors can have multiple instruction executing units or cores. Such processors can also be implemented as one or more software modules in programming languages such as, for example, Java™, C++, C, assembly, a hardware description language, or any other suitable programming language. A processor according to some embodiments can include media and computer code (also can be referred to as code) specially designed and constructed for the specific purpose or purposes. In some embodiments, the processor(s) 128 can support standard HTML, and software languages such as, for example, Javascript, Javascript Object Notation (JSON), Asynchronous Javascript (AJAX).

In some embodiments, a processor can be, for example, a single physical processor such as a general-purpose processor, an ASIC, a PLD, or a field programmable gate array (FPGA) having a single processing core or a group of processing cores. In some embodiments, a processor can be a group or cluster of processors such as a group of physical processors operatively coupled to a shared clock or synchronization signal, a shared memory, a shared memory bus, and/or a shared data bus. In other words, a processor can be a group of processors in a multi-processor computing device. In some embodiments, a processor can be a group of distributed processors (e.g., computing devices with one or more physical processors) operatively coupled one to another via a communications network. Thus, a processor can be a group of distributed processors in communication one with another via a communications network. In some embodiments, a processor can be a combination of such processors. For example, a processor can be a group of distributed computing devices, where each computing device includes a group of physical processors sharing a memory bus and each physical processor includes a group of processing cores.

Processor(s) 128 are also operatively coupled to memory 126. Memory 126 can be, for example, a read-only memory (“ROM”); a random-access memory (“RAM”) such as, for example, a magnetic disk drive, and/or solid-state RAM such as static RAM (“SRAM”) or dynamic RAM (“DRAM”); and/or FLASH memory or a solid-data disk (“SSD”). In some embodiments, a memory can be a combination of memories. For example, a memory can include a DRAM cache coupled to a magnetic disk drive and an SSD.

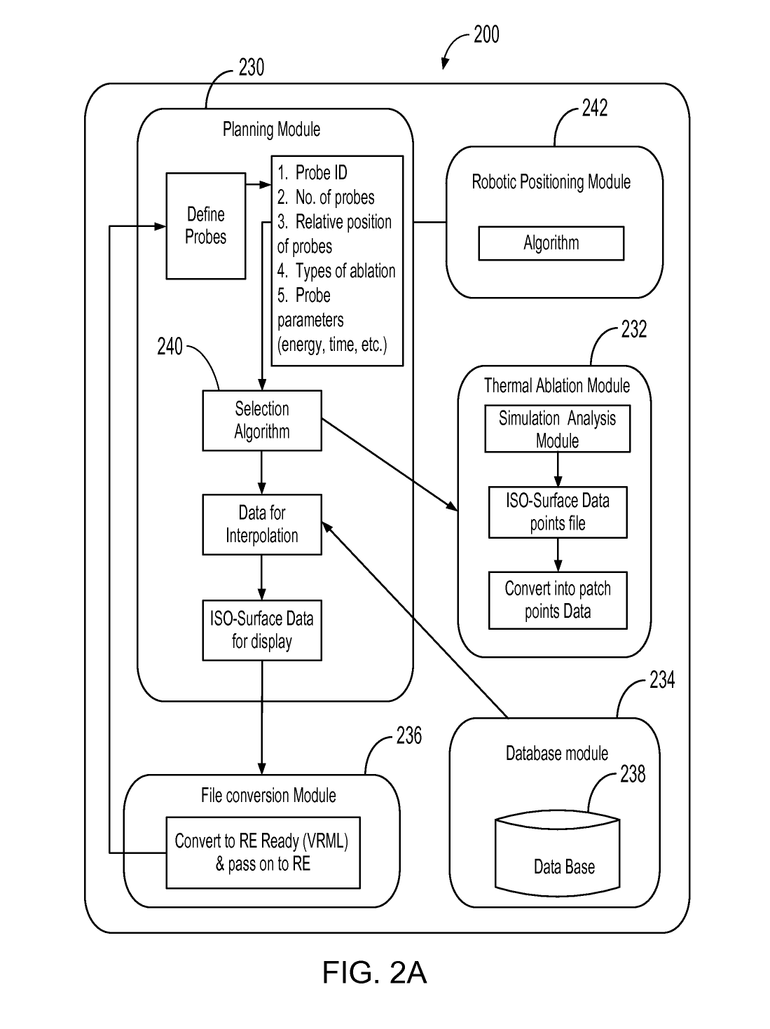

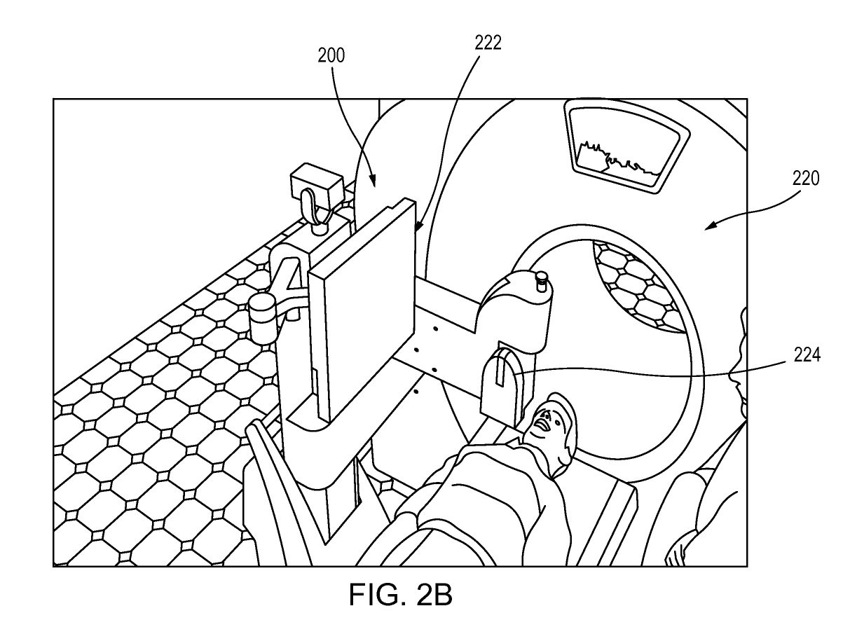

The planning station 100 can include a planning module, a robotic positioning module, a thermal ablation module, an ablation volume data module and a file generation module (each shown in FIG. 2A but not shown in FIG. 1), as described in more detail below. Each of the planning module, thermal ablation module, ablation volume data module and VRML module can include one or more processors and/or one or more memory components as described above. The planning station 100 can also include a database (not shown in FIG. 1) that can include a processor(s) and/or memory(ies) as described above.

The planning station 100 can be in electrical communication with the imaging device 120 and the positioning device 124. The planning station 100 can be coupled to the imaging device 120 and the positioning device 124, or can communicate via a wireless connection with one or both. The imaging device 120 can be for example, a computed tomography (CT) imaging device, a magnetic resonance imaging (MRI) device, or the other imaging device. The imaging device 120 can include, for example, a cradle that is movable over a table on which the patient can be disposed during an interventional procedure as described herein. The imaging device 120 can also be in electrical communication with the positioning device 124 (either coupled thereto or via a wireless connection) and can interface with the planning station 100 and the positioning device 124 with, for example, a Digital Imaging and Communications in Medicine (DICOM) standard, such as DICOM 3.0.

The positioning device 124 can be, for example, an apparatus that can be used to determine an angle and depth of insertion of an interventional tool to be used during an interventional procedure. An example of a positioning device 124 is described in U.S. Patent Application Publication No. 2008/0091101 (“the '101 publication”), the disclosure of which is hereby incorporated herein by reference in its entirety (see also, FIG. 2B, which illustrates another example of a positioning device 224 that can be used in conjunction with a CT imaging device 220 and a planning station 200 as described herein). As described in the '101 publication, the positioning device 124 can be used in conjunction with the imaging device 120 to calculate an angle and depth of insertion of an interventional tool, such as interventional tool 122, into a patient to treat an area of interest (e.g., a tumor). The positioning device 124 can position a tool guide of the positioning device 124 at a designated location relative to the patient and a physician can then use the tool guide to position accurately the interventional tool 122 for manual insertion into the patient.

As discussed above, the planning station 100 can be used to generate a plan for an image-guided interventional procedure and provide a simulation of the plan viewable on the display device 122. In use, a physician can image an area of interest on a patient to be treated using the imaging device 120. A set of images from the imaging device 120 can be used by the planning station to create a 3D reconstruction of the image set. The planning station 100 can includes segmentation and visualization tools to allow the user (e.g., physician) to interactively segment and visualize relevant anatomical structures in 2D, MPR (multi-plan reformatting), and/or 3D formats. Thus, the user can segment an area of interest, such as, for example, a tumor volume. The images can be viewed by the user on the display device 122 and the user can select a target tissue within the area of interest to be treated.

The planning station 100 can store data associated with various interventional tools (e.g., applicators, needles, etc.) within its memory 126. The planning station 100 can also include one or more databases (not shown in FIG. 1) that can include a memory for storing data, such as, for example, data associated with various interventional tools and data associated with thermal models associated with various interventional tools. Based on the image-segmented image data, the user can select an appropriate interventional tool 126 to perform an interventional procedure on a selected target tissue within the area of interest on the patient. For example, to perform an ablation procedure on an area of interest within a patient identified with the image data provided by the imaging device 120, the user can select one or more ablation tools included in a list of tools stored in the planning station 100. The user can then select desired ablation parameters (e.g., energy level and time period for ablation) to use to generate a visual analysis of an ablation volume of the tumor to be treated using the selected ablation needle(s). Based on the imaging data, the selected area of interest (e.g., tumor) to be treated, and the selected interventional tool(s) (e.g., ablation needles), the user can select a desired insertion point and target point at which the interventional tool(s) should be placed to achieve a desired outcome.

The planning station 100 can then generate a simulation viewable on the display device 122 of a treatment plan to treat the target tissue using the selected interventional tool(s), and the desired insertion parameters. The simulation can include superimposed images of the first interventional tool and the second interventional tool and the area of interest to be treated. For example, in a procedure using multiple interventional tools, the simulation can show a first interventional tool disposed at a first location within the body of the patient and a second interventional tool disposed at a second location within the body of the patient. Although two interventional tools are described, it should be understood that a different number of interventional tools can be used (e.g., three, four, five, etc.). The planning station 100 can determine if the desired insertion point and target point selected by the user can be used without interference between interventional tools and/or can be achieved with the particular positioning device 124 to be used to place the interventional tools. Thus, the planning station 100 can generate a treatment plan that provides for collision avoidance between multiple interventional tools to be used during the interventional procedure. In some embodiments, the planning station 100 can generate a treatment plan that also provides for collision avoidance of organs and other tissue within the patient.

The simulation can also show the application of a first amount of energy from the first interventional tool and the application of a second amount of energy from the second interventional tool in the body of the patient. The planning station 100 can also generate a thermal model of the thermal effect collectively produced on the tissue of patient (e.g., including the target tissue and surrounding tissue), produced by the first interventional tool and the second interventional tool based on the simulation. In other words, the thermal model generated by the planning station 100 can simulate the combined thermal effects on the patient's body, and the target tissue, of treatment with multiple interventional tools (e.g., multiple ablation needles). Based on the simulation and the thermal model of the ablated tissue, the user can determine if any adjustments need to be made to the plan before proceeding to the actual interventional procedure. The thermal effect on the tissue of the patient produced by the interventional tool may result from electromagnetic fields produced by the tool (e.g. in the radio-frequency (RF) or microwave spectrum), infrared energy, cryogenic cooling, heated fluid, etc.

After the simulation is completed and a treatment plan has been generated, the user can proceed to execute the treatment plan during an image-guided interventional procedure. For example, the treatment plan can be provided to the positioning device 124, which can use the treatment plan data received to position a robotic arm of the positioning device relative to the patient to provide for the desired insertion position of the selected interventional tool(s). As discussed above, the positioning device 124 can position a tool guide (also referred to herein as “end effector”) of the positioning device 124 at the determined location relative to the patient and a physician can use the tool guide to position accurately the interventional tool 122. In some embodiments, the physician manually inserts the interventional tool 122 into the patient.

In some embodiments, during an interventional procedure using multiple interventional tools, the user can check or verify the position of the interventional tools at various points during the procedure. The user can also check the progress of treatment during the procedure. For example, after insertion of the first interventional tool into the patient according to the treatment plan, the user can image the area of interest to verify that the position of the first tool is at the desired location relative to the target tissue to be treated. If the position of the first interventional tool is determined to be satisfactory, the user can proceed with the treatment plan (e.g., insertion of the second interventional tool and applying thermal energy, etc.). If the position of the first interventional tool is deemed to be at an undesirable location, the user can request that the planning station 100 use the image data to generate a new or revised treatment plan and a simulation thereof. In another example, the user can image the area of interest after applying a first amount of energy from the first interventional tool and verify if the amount and location of treatment is as desired for that stage in the procedure. If not, the user can request that the planning station 100 use the image data to generate a new or revised treatment plan and a simulation thereof. The new treatment plan can, for example, include a different amount of energy (i.e. different power level and/or duration of treatment) to be applied by one or more of the interventional tools.

FIG. 2A is a schematic illustration of an embodiment of a planning station 200. The planning station 200 includes a planning module 230, a robotic positioning module 242, a thermal ablation module 232, an ablation volume data module 234 and a file generation or conversion module 236. The planning station 200 can include one or more processors (e.g., processor 128) and one or more memory components (e.g., memory 126) as described above for planning station 100 and each of the planning module 230, robotic positioning module 242, thermal ablation module 232, ablation volume data module 234 and file generation module 236 can also include one or more processors and/or one or more memory components as described above. The planning station 200 also includes a display device 222 and can be in electrical communication with an imaging device 220 and a positioning device 224, as shown in FIG. 2B. The planning station 200 can be used during an image-guided interventional procedure as described above.

Thermal ablation of a solid tumor in a tissue with, for example, RF energy can be accomplished by using a probe (e.g., an ablation needle) inserted into the tissue under the guidance of a suitable imaging modality, such as, for example, a CT imaging device. The extent of the ablation can be significantly reduced by heat loss from capillary perfusion and by blood flow in a large vessel in the tissue. A mathematical model can be presented to a user that shows the thermal processes that occur during ablation of a tissue near a large blood vessel, and which should not be damaged. Temperature distribution dynamics are described by the combination of a 3D bioheat transport in tissue together with a 1D model of convective-dispersive heat transport in the blood vessel. The objective is to determine how much of the tissue can be ablated without damaging the blood vessel. This can be achieved by simulating the tissue temperature distribution dynamics and by determining the optimal power inputs from the ablation needles so that a maximum temperature increase in the tissue is achieved without inducing tissue damage at the edge of the large vessel.

The thermal ablation model can simulate the ablation process based on the following parameters that can be input into the planning station 200 by a user (e.g., physician):
Probe type—entered as a probe ID;
Number of probes to be used for a particular treatment plan;
Relative position of the probes with respect to the first probe (assuming the first needle is the null vector);
Type of ablation;
   Simultaneous ablation—all probes are energized at the same time;
   Sequential ablation—probes are energized one after the other (e.g., the same probe type can be used multiple times); and
Probe parameter settings—based on the type of probe as sequential values, where the sequence is predefined for each probe model or type.

The thermal ablation model can be integrated with the planning software within the planning station 200. The planning software can supply the input parameters to the selection algorithm 240 for algorithm selection and input to the thermal ablation module 232. The thermal ablation module 232 can incorporate regions of tissue coagulation that are consistent with ablation system manufacturers' specifications, and can generate isothermal surface data associated with various discrete power and time values as point clouds for each setting can be stored within the thermal ablation module 232. Isothermal surfaces for other power and time values can be generated by interpolation between stored isothermal surface data sets, or can be generated by the thermal ablation module 232.

To plan for an interventional procedure, the user (e.g., physician) can first select a desired entry point and target point for each selected probe (e.g., ablation needle) to be used during the interventional procedure. For example, the user can select/enter the probe(s) parameters as described above. First, the user can select a first probe to be used and a 3D image can be generated to show the first probe superimposed with an image of the region of interest to be treated within the patient. The user can then position the probe shown in the image on the display device 220 (e.g. by a “click and drag” operation) to a desired entry and target location. The planning station 200 can then perform several checks on the indicated position of the probe, e.g. to determine if the selected entry and target points are within a range of reach of the robotic arm of the positioning device 224, whether the probe will pass through a volume within the patient's body that the user has identified as a volume not to be penetrated (such as an organ), whether the robotic arm would interfere with patient's body when placing the probe in the indicated position, whether the entry point is accessible to the robotic arm (e.g. if the selected entry point is on a portion of the user's body that is obstructed, such as being adjacent the bed), whether the distance between the target point and the entry point exceeds the length of the needle (allowing for a specified “dead space” between the handle end of the needle and the patient's skin). For example, the robotic positioning module 242 can use a robotic positioning algorithm to check for interference between the skin surface of the patient and the robotic arm. A 3D image is generated to ensure that the robotic arm orientation is in compliance with the robot arm position with respect to the patient's body. A check can also be performed to verify a minimum gap between the robotic arm and skin surface at any given point during the planned interventional procedure. If the position of the first probe is not acceptable (e.g., there is an interference), the user will be alerted to change the desired probe path. For example, the image of the probe can be color coded to indicate whether or not the probe can be placed in the indicated position without violating any of the conditions identified above, e.g. green if acceptable, red if not acceptable. The user can then reposition the first probe shown in the 3D image on the display device 220. This process can continue until the planned insertion and target points selected for the first probe are acceptable.

If a second probe is to be used during the interventional procedure, the user selects the desired parameters for the second probe as described above for the first probe. The planning station 200 can next produce a 3D image to show the placement of the first probe and the desired placement of the second probe superimposed with an image of the region of interest to be treated within the patient. The robotic positioning module 242 can check for interference with the robotic arm and with the patient as described above for the first probe, and also check for interference between the selected path for the first probe and the selected path for the second probe. For example, checks can be performed to determine if a sufficient gap is present between the portions of first probe and the second probe that are within the patient, and between the portions of the probes that are outside of the patient's body (i.e. whether the handles of the probes are interfering with each other), and whether the robotic arm would engage a previously-placed probe when placing the current probe. As described for the first probe, if the selected entry and target points for the second probe are not acceptable, the user can move the second probe to a different location within the 3D image and this process can continue until an acceptable position for the second probe is achieved. If a third probe is selected, a check is performed to determine compliance with specified conditions, such as those discussed above. This process is performed for each probe to be used during the planned interventional procedure.

After the interference determinations have been performed, an optimal sequence to insert the probes can be determined. For example, the user may select a probe 1, probe 2, and probe 3 (in this order), and the robotic positioning module 242 may reorder the probes for an optimal insertion sequence to prevent interference (e.g., probe 2, probe 1 and then probe 3). After the optimal sequence has been determined, and the planning process has been completed, the robotic arm can be positioned during the interventional procedure as per the angles calculated for the probes.

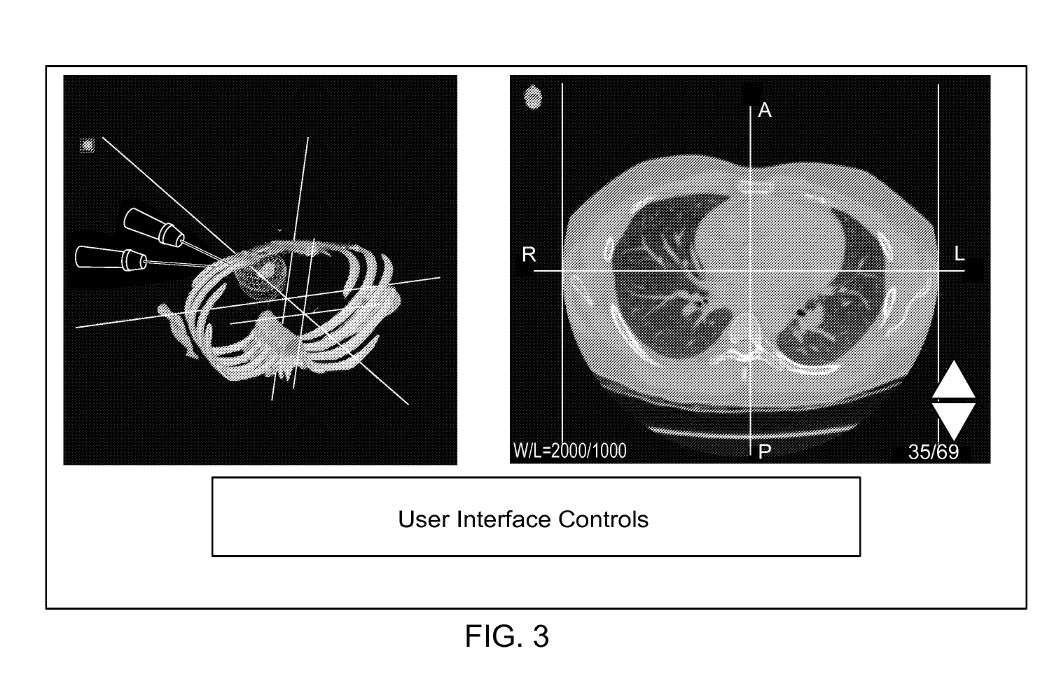

After determining the optimal planned entry and target points for the selected probes, the thermal ablation module 232 can be used to create the thermal models (discussed above). The thermal ablation models can emulate heating in homogeneous tissue as specified by the manufacturer of the particular ablation system/tool being used for an interventional procedure. The thermal models can produce data that are used to develop embedded geometries of heated tissue volumes that are consistent with manufacturers' specifications. In some embodiments, these embedded geometries are not used for patient-specific ablation treatment planning capabilities on the positioning device (e.g., positioning device 124), but rather non-patient-specific lesion targeting for ablation applications (such as radio frequency ablation (RFA) applications). See, for example, FIG. 3, which is a schematic illustration of a display of a targeted lesion to be treated.

Ablation device geometries and materials can be determined from product specifications for the particular ablation tool. Tissue dielectric and thermal properties can be chosen, for example, from measurements recorded in peer-reviewed published journals. Thermal simulation modeling can be performed, for example, using COMSOL Multiphysics Modeling and Simulation Software, including the “Heat Transfer” and “RF” modules thereof or using other suitable finite element analysis or other techniques. The thermal simulation model can predict, for example for RF ablation, real-time coupling of the electromagnetic fields to the tissue, thereby causing a temperature increase related to the field intensity.

Thus, the thermal ablation module 232 can generate iso-surface plots for a given probe (e.g., ablation needle), at a given time, and at a given power level for that probe. For example, an iso-surface plot for such parameters of the probe can be generated for a temperature of 67 degrees C. (i.e., a temperature at which tissue necrosis can be considered to occur). The iso-surface plots can be stored in a database (e.g. database 238) and can be made available to a user when planning a particular treatment plan. When a user (e.g., a physician) is generating a particular treatment plan, the user inputs the various parameters for a selected probe. If an iso-surface plot is available in the database that matches, the planning module 230 can use the stored iso-surface plot to generate a thermal simulation of the treatment plan. If an iso-surface plot is not available for the particular probe (e.g., for a particular time period at a particular power level), the ablation volume data module 234 can either use the iso-surface plots stored in the thermal ablation module 232 to interpolate and generate a new iso-surface plot associated with the desired parameters or have the thermal ablation module 232 generate a new one. For example, the planning module 230 can use four iso-surface plots (i.e., one having the closest time greater than the desired time, one having the closest power greater than the power desired, one having the closest time less than the desired time, and one having the closest power less than the desired power) for interpolation.

As discussed above, the planning module 230 can interpolate iso-surface data for RF ablation that is produced by the thermal ablation module 232. Isothermal surface data is available for various discrete power and time values as point clouds (e.g., as set of vertices in a three-dimensional coordinate system) for each setting. In this manner, interpolation is a method of constructing new data points within the range of a discrete set of known data points. The planning module 230 can simplify the data, store the data into database 238, and can be used in a selection algorithm 240 based on the input parameters described above.

In one example thermal simulation model, the ablation system chosen is a Covidien Cool-tip Single (Active 3 cm electrode). It should be understood that other ablation systems/tools can alternatively be used. The ablation applicator modeled is a 17 gauge Cool-tip single electrode with a 30 mm radiating element that employs 20 C water-cooling and is energized with power set at 25, 50, 100 and 200 watts at steady state. The tissue load is modeled as healthy bovine ex-vivo liver tissue as identified in manufacturers' specifications, with blood perfusion, with the following properties.

| | |
|---|---|
| Property | Value/Units |

| | | |
|---|---|---|
| electrical conductivity | 0.3 | s/m |
| thermal conductivity | 0.512 | W/(m · K.) |
| density | 1060 | kg/m³ |
| thermal density | 3600 | J/(kg K.) |
| blood density | 1000 | kg/m³ |
| blood specific heat capacity | 4180 | J/(kg K.) |
| blood perfusion rate | 6.4 × 10⁻³ | m/s |
| arterial blood temperature | 310 | K. |

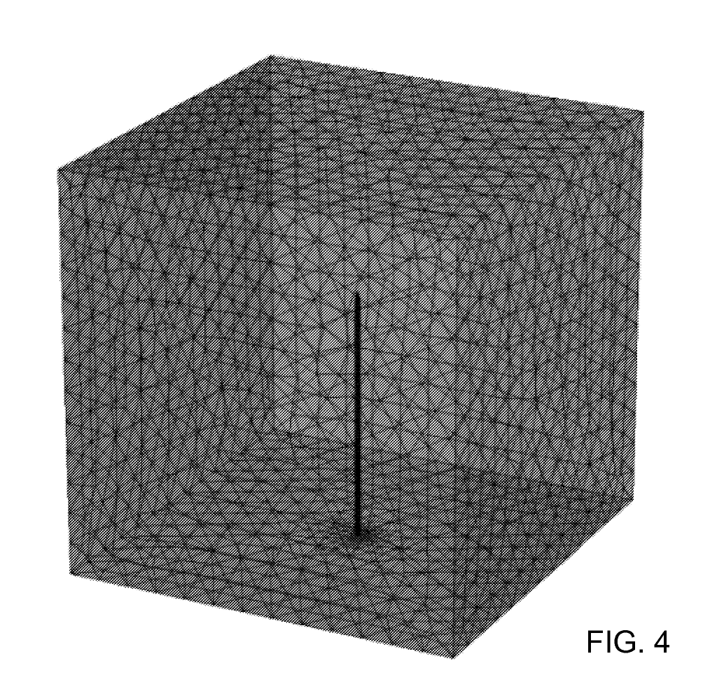

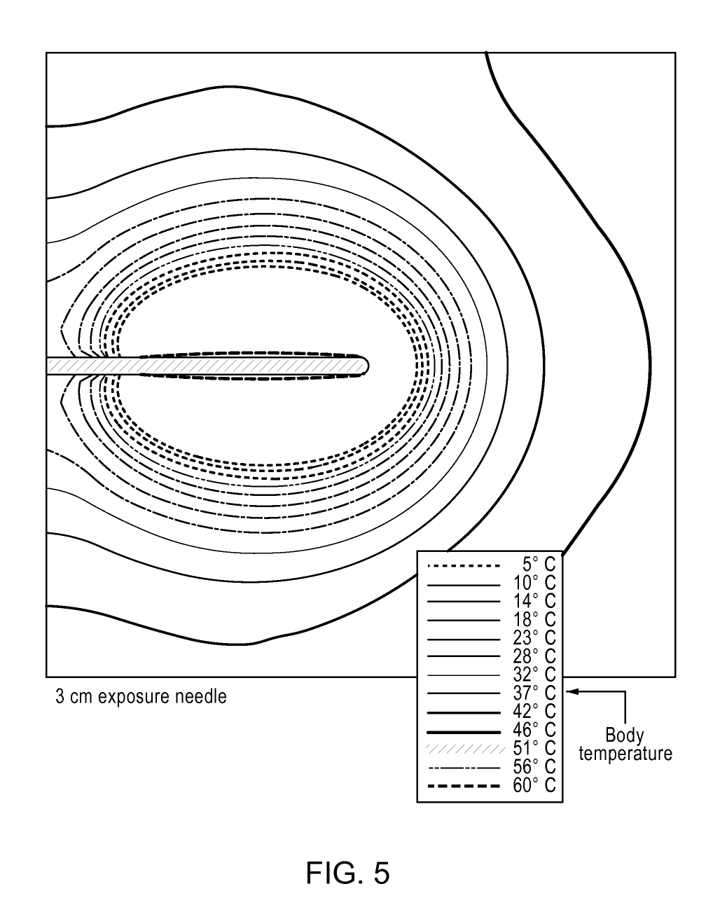

In this example, all dimensions are in meters, therefore, the tissue load dimensions are 0.12 m×0.12 m×0.12 m. The center of the ablation site for all the ablations is at the centre of a simulated mesh tissue block and radiating element (e.g., ablation needle), as shown in FIG. 4. Small errors in the shape can be due to the approximation of the conical end of the applicator, but these errors are negligible for the purposes of this example. The model produces ablation regions (see, FIG. 5) that are comparable with the published cool-tip on Valleylab's website (see, http://www.cool-tiprf.com/ablation.html).

The example illustrates a simulated thermal model for a single probe (e.g., FIG. 4). When multiple probes are to be used for a particular interventional procedure, the planning station 200 could employ one of two techniques. The planning station 200 could specifically model the effects of the multiple ablation tools, e.g. using the technique described above for a single tool. Alternatively, the planning station 200 could generate a simulation of the tissue ablation by aggregating, or interpolating, the thermal effects modeled for each of the multiple ablation tools to be used. First, the ablation volumes can be determined for each of the multiple ablation tools. Then, each of the ablation volumes can be placed in three-dimensional space based on the ablation tool placement specified by the user. Next the volume(s) defined by the intersection(s) of the individual ablation volumes can be determined. The tissue temperature at each point in the intersection volume(s) can be calculated based on the temperature gradients produced at the point by each of the individual ablation tools. Then, the planning station 200 can calculate the location of the geometric center of the combined intersecting volume(s) and the rays from the geometric center along which the tissue temperature needs to be determined. Next, based again on the temperature gradient produced by each ablation tool at points along each of the rays, the planning station 200 can determine the point on each ray at which the desired temperature (e.g. the 67 degrees C. temperature at which tissue necrosis can be considered to occur) would be produced by the ablation tools. These points collectively define the ablation iso-surface for the desired positions and energies of the ablation tools.

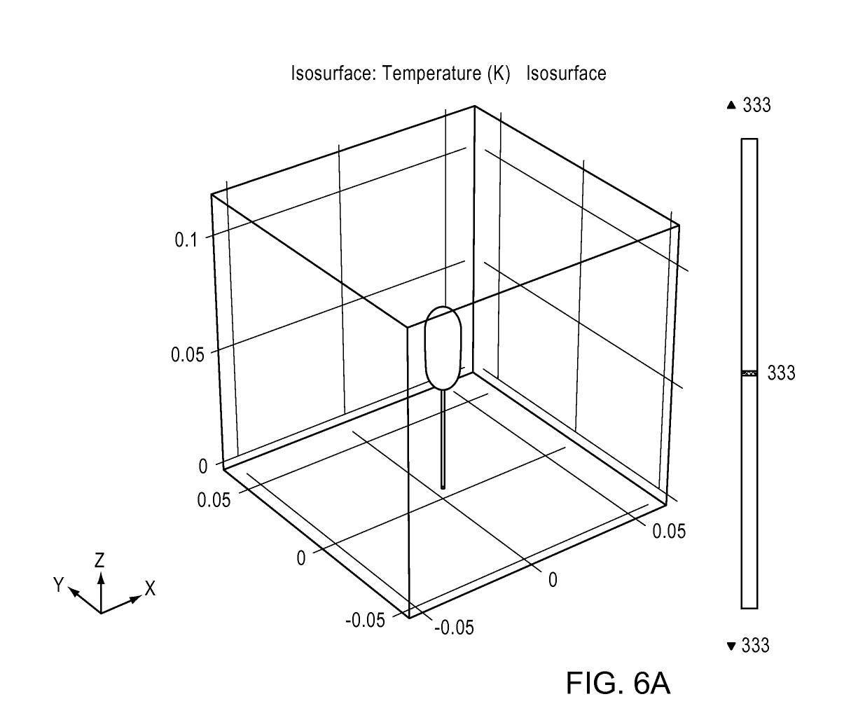

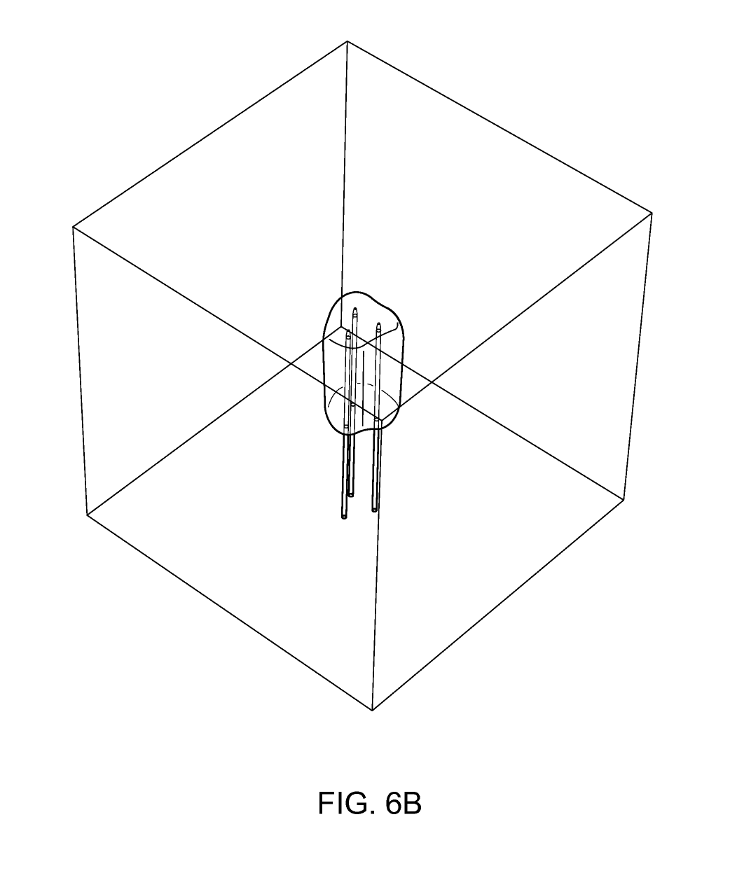

The modeled data plots can include, for example, slice plots (not shown), contour plots (not shown) and surface plots, such as, the example surface plot shown in FIG. 6A for the single applicator with a 30 mm radiating element described above with power applied at 100 W for a duration of 600 seconds. In FIG. 6A, the temperature T=60 degrees C. is shown as 273+60=333 K. Data files for each plot can be included as raw data in the form of x-coord, y-coord, z-coord, temperature (K) and labeled accordingly in, for example, an Excel worksheet(s) stored in a memory of the thermal ablation module 232. The surface plots (e.g., FIG. 6A) can be used in the interpolation algorithm of the ablation volume data module 234 described below.

In some cases, when multiple ablation tools are to be used for an interventional procedure, the modeled plots can be used to account for spacing between the electrodes (e.g., the ablation tool tip). For example, some manufacturers recommend that the spacing between electrodes be no greater than 20 mm to avoid untreated regions of tissue. With spacing between electrodes, for example, of 10 mm, the ablation region can be more uniform with the regions of untreated tissue being eliminated or significantly reduced. FIG. 6B illustrates an example surface plot for three probes each having a 30 mm radiating element and with power applied at 200 W for a duration of 600 seconds. The spacing between the probes in this example is 10 mm.

The following describes an example interpolation of iso-surface data for RF ablation. In this example, the point cloud for a particular discrete power setting Pₖ, t₁ is SX(Pₖ, t₁), k, 1=1,2,3,4. It can be assumed for this example that the points are described in a coordinate system with the origin at a center of the ablation needle. In this example method of interpolation, the solution approach can be as follows:
1. Segment the point cloud set into octants.
2. Convert the point cloud to a spherical coordinate (θ, φ, r) format with origin at the needle center.
3. Tile the surface described by the point cloud with respect to θ, φ to any order of resolution required.
4. Approximate the value of the surface at each voxel as the mean of all the points contained within it.
5. Fit a parametric cubic surface across the power and time values.
6. Use this parametric surface to estimate the coordinates at any given power and time value.

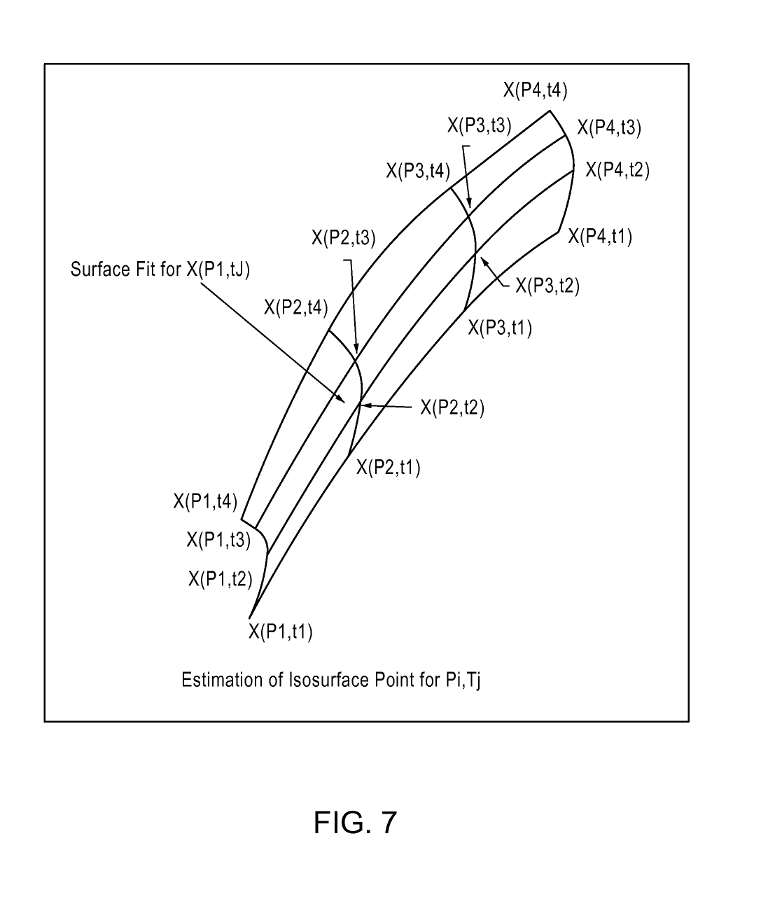

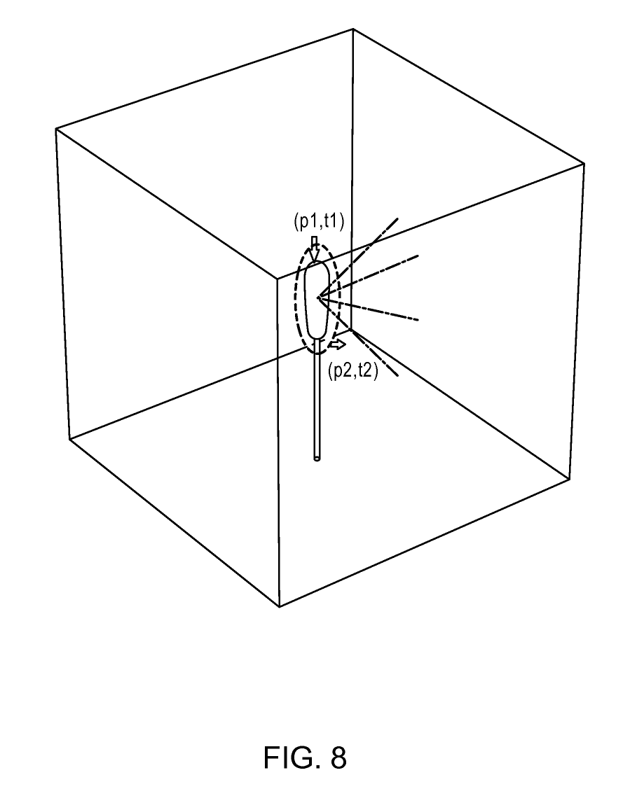

As shown in FIG. 7, each control point X(Pᵢ, tⱼ) represents the estimated point on the surface fit for power Pₖ and t₁. Using these control points, the cubic parametric surface can be estimated as a function of two parametric variables u (along Power) and v (along time). For any given power, time value Pᵢ and tⱼ, the uᵢ and vⱼ parameter corresponding to this power level can be computed and an estimate of the point on the iso-surface X(Pᵢ, tⱼ) using the parametric surface can be determined as shown in FIG. 8.

The ablation volume data module 234 can cut the region into predefined angles—the smaller the angles, the greater the accuracy. After cutting the region, the ablation volume data module 234 uses the database 238 to store the data. The values that are passed to and stored in the database 234 can include, for example, the Probe Name, Manufacturer, Temperature, Energy(p), Time(t), the angles and the coordinates of each point in the point cloud

The database 238 can include stored information about various interventional tools that can be used to perform the desired treatment. The database 238 can also include a 3D model of each of the interventional tools in a format (.step, .stl, .VRML etc.) readable into a 3D rendering engine, the physical dimensions (e.g., length, diameter, variable ablation parameters with range of operation) and the ablation models generated for the selected probe on a homogeneous tissue model using discrete settings as described above (e.g., time intervals of 5, 10, 15, 20 minutes and energy settings of 50, 100, 150, 200 watts and distance between probes in various combinations). The database 238 can also include ablation models generated for multiple probes (e.g., illustrating the collective thermal effect of multiple probes being used for an ablation procedure).

The database 238 can be a structured query language (SQL) server and use an application programming interface (API) to access the database 238 for both read and write functions. The database 238 can include a probe data table that includes various information about the various available interventional tools (e.g., ablation probes). For example, the database 238 can include the following information about known interventional tools:

1. Probe.probeId

2. Energy (Power)

3. Time

4. Point cloud data for the above parameters

The planning station 200 also includes a selection algorithm 240 (see, FIG. 2). The selection algorithm 240 can use input parameters received from the planning module software. The input parameters can include, for example, the following:

1. Probe ID;

2. No. of probes;

3. Relative position of probes;

4. Types of ablation; and

5. Probe parameters (energy, time, etc.).

The selection algorithm 240 can include the following functionalities to return the thermal model surface plots:
1. Can use the probe detail to get the information from a probe data table in the database 238.
2. If the algorithm finds the exact values for a desired time and energy, it returns that set of surface points.
3. If not, the algorithm
   a) finds the closest four models (e.g., for four available probes within the database) to the selected point and interpolates the models; or
   b) generates a new model using the equations.

To get a value (pₖ, tₖ) which is in between the (p₁, t₁) & (p₂, t₂), the algorithm will use the (p₁, t₁) and (p₂, t₂) at all (φ, Q) values. For the selected probe and probe parameters, all the X1, y1, Z1, Delx, Dely and Delz available for the Q, φ angles will be read from the database 238 for both (p₁, t₁) and (p₂, t₂). Then the system can calculate the value for the pk,tk passed for all the angles to get the ISO surface data points.

X₀₀−Isosurface coordinate<x,y,z>at Power value p₁ and time value t₁

X₀₁−Isosurface coordinate<x,y,z>at Power value p₁ and time value t₂

X₁₀−Isosurface coordinate<x,y,z>at Power value p₂ and time value t₁

X₁₁−Isosurface coordinate<x,y,z>at Power value p₂ and time value t₂

To determine Isosurface Coordinate <x,y,z> at intermediate power value pₖ,tₖ.

1. Determine u and v

u=(Pᵤ−P₀)/(P₁−P₀)

v=(Tᵥ−T₀)/(T₁−T₀)

2. Determine intermediate intercepts along P

Xᵤ₀=X₀₀+u*(X₁₀−X₀₀);

Xᵤ₁=Xₒ₁+u*(Xₒ₁−X₁₁);

3. Determine final point as:

Xᵤᵥ=Xᵤ₀+v*(Xᵤ₁−Xᵤ₀);

The equations above can be applied to every coordinate of X (i.e. <x,y,z>) separately. These data points can then be connected to form a surface/region based on the angles. The file generation module 236 will take the inputs of the ISO surface data points and write them in VRML format. The planning software within the planning module 230 can supply the input parameters to the selection algorithm 240 and display the output of a rendering engine of a display device (e.g., display device 122).

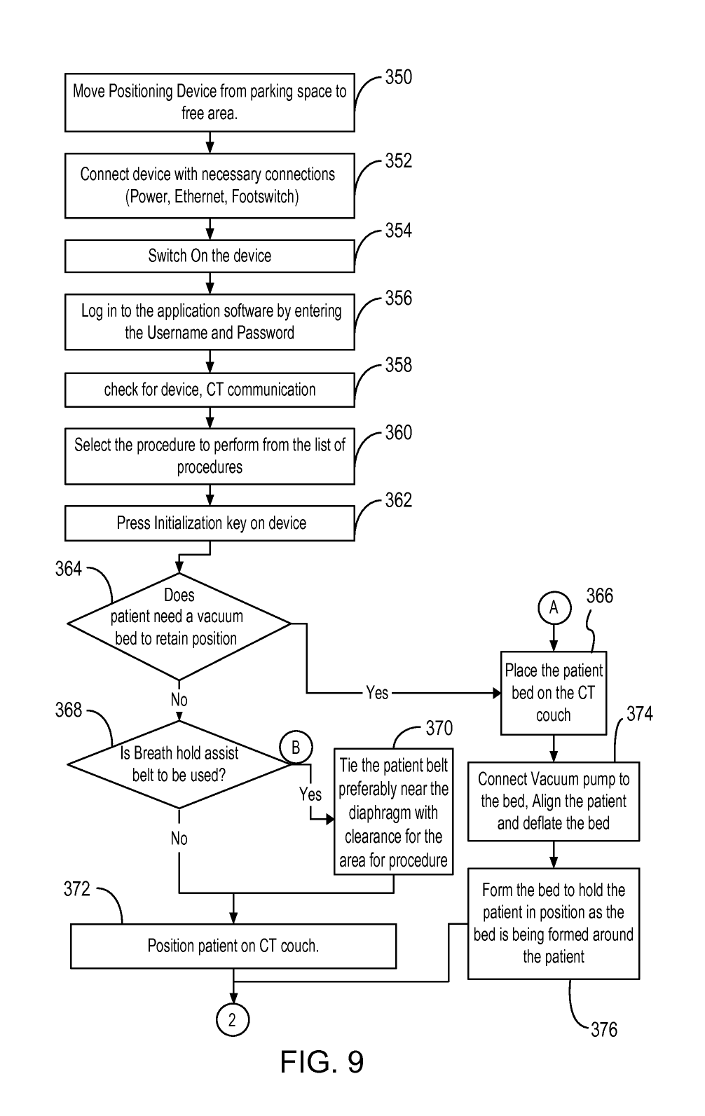

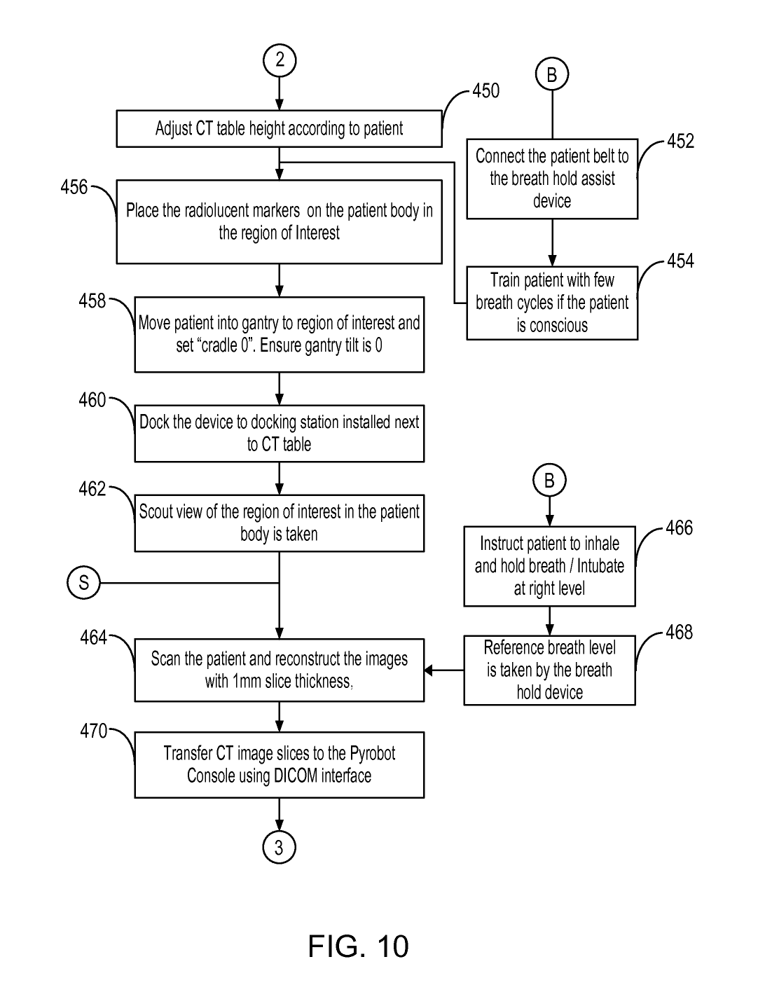

FIGS. 9-18 are each a flow chart illustrating a stage or portion of an example method of performing an ablation procedure including the use of a planning station, positioning device, imaging device, and display device as described herein. As described in FIGS. 9-18, when reference is made to a button or switch on a user interface of a display device or other component, such a button or switch can alternatively be presented as a tool on a touch screen or a pull-down menu on a display device that can be clicked. FIG. 9 is a flow chart illustrating a method for performing a set-up procedure to prepare the positioning device and patient for an image-guided interventional procedure (e.g., an ablation procedure), and FIG. 10 is a flow chart illustrating a method of preparing an imaging device and the patient for images to be taken, and imaging a region of interest on the patient.

As shown in FIG. 9, at 350 the positioning device is moved from a location where it is being stowed to a desired location to perform a procedure in proximity to a CT imaging device. At 352, the necessary connections (e.g., power, Ethernet, footswitch, etc.) are made to allow operation and communication of the positioning device. At 354, the positioning device is switched on, and at 356, the user can log in to the application software, for example, by entering a username and password. At 358, communication with the CT scanner can be verified. At 360, a procedure to be performed can be selected from a list of procedures stored in a database of the system. At 362, an initialization key on the positioning device is pressed. In some embodiments, the device does a partial initialization and positions to predefined X, Y, Z, A, B values (e.g., X, Y and Z are the axial positions along the linear degrees of freedom associated with the robotic arm and A, B are rotational positions about the rotational degrees of freedom associated with the robotic arm). At 364, a determination can be made as to whether the patient will need a vacuum bed to retain the position of the patient. If a vacuum bed is needed, the patient bed is placed on the CT couch at 366, and at 374 a vacuum pump is connected to the bed, the patient is aligned and the bed is deflated. At 376, the bed is formed to hold the patient in position as the bed is being formed around the patient.

If a vacuum bed is not needed, at 368 a determination can be made as to whether the patient will need a breath hold assist belt. If a breath hold assist belt is needed, at 370, the belt is tied to the patient near the patient's diaphragm with clearance for the procedure area. In addition, additional procedures are to be followed later in the method if a breath hold assist belt is used, as shown at circle B in FIG. 10. At 372, the patient can be positioned on the CT couch. Optionally, there may be a visual indicator (such as an adhesive label) on the CT table that indicates the limit of the range of the positioning device, in which case the patient is positioned on the CT table so that the region of interest is within the limit of the range.

As shown in FIG. 10, at 450, the height of the CT table can be adjusted to accommodate the particular patient. If at 368 it was determined that a breath hold belt was needed, the patient belt is connected to a breath hold device at 452, and at 454, if the patient is conscious, the patient can be trained on performing breath cycles. At 456, radiolucent markers can be placed on the patient's body in the region of interest. For example, in some embodiments, three radiolucent markers are used if, for example, no intra-operative registration is needed. In some embodiments, four or five markers are used. At 458, the patient can be moved into the gantry of the CT imaging device, and at 460, the CT device can be docked or coupled to a docking station of the positioning device.

At 462, a scout view of the region of interest in the patient can be taken. At 464, the patient can be scanned and images reconstructed with, for example, 1 mm slice thickness. In some embodiments, it may be desirable for the image offset to be 0,0 during the image reconstruction. If the breath hold assist device is used (from 350 in FIG. 9), prior to the image reconstruction, the patient can be instructed to inhale and hold breath at 466 (or, if the patient is anesthetized and therefore intubated, the external user can control the breath as desired), and the referenced breath level can be taken by the breath hold device at 468. After the image reconstruction, the CT image slices can be transferred to the positioning device console using, for example, a DICOM interface.

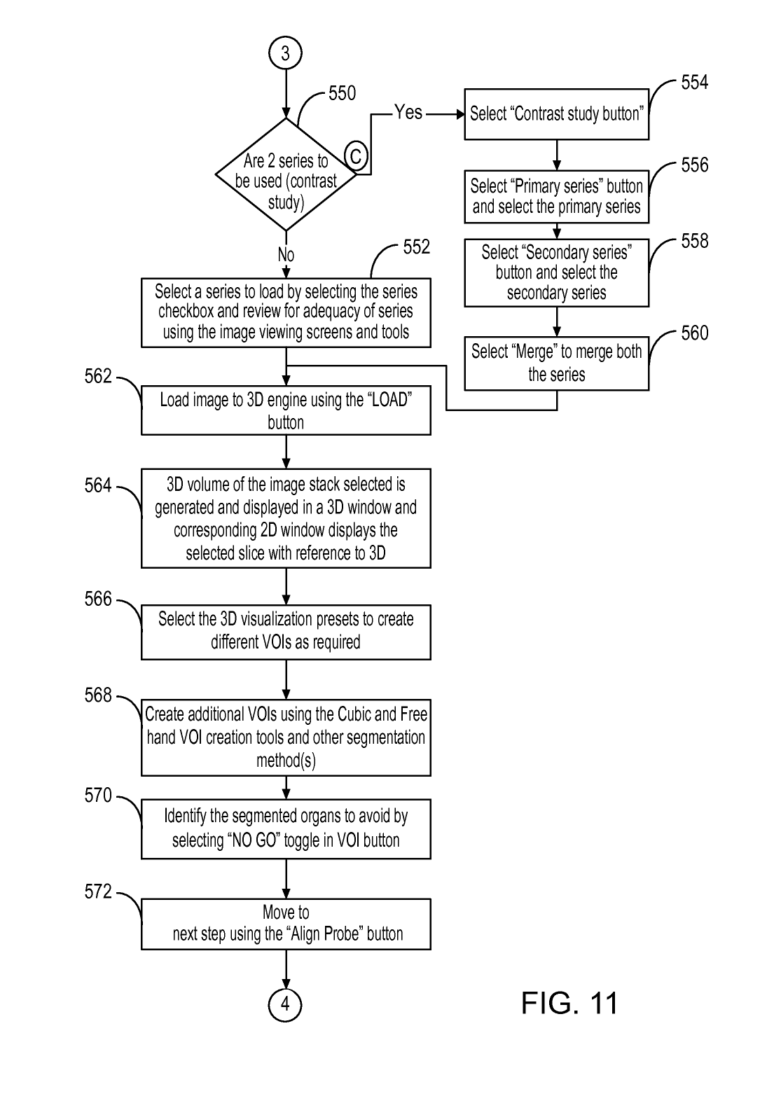

FIGS. 11-16 each illustrate a portion of a method of generating a plan for execution of an image-guided interventional procedure and a simulation of such plan, according to an embodiment. For example, a planning station as described herein can be coupled to, and in communication with, the positioning device described above with reference to FIGS. 9 and 10. At the planning station, at 550, a decision can be made as to whether two series of images are to be used in a contrast study. At 552, if only a single image series is to be used, a series to load can be selected, for example, by selecting a series checkbox (e.g., on a user interface screen). For example, the planning station and/or display device can have user interface tools to allow the user to navigate through the planning process. Image viewing screens (e.g., on the display device) and tools can be used to review the images for accuracy. If a contrast study is to be performed, at 552, a contrast study button/switch on the user interface of the planning station can be selected. At 556, a primary series button can be selected at 556, and a secondary series button can be selected at 558. The two selected image series (e.g., primary and secondary) can then be merged at 560.

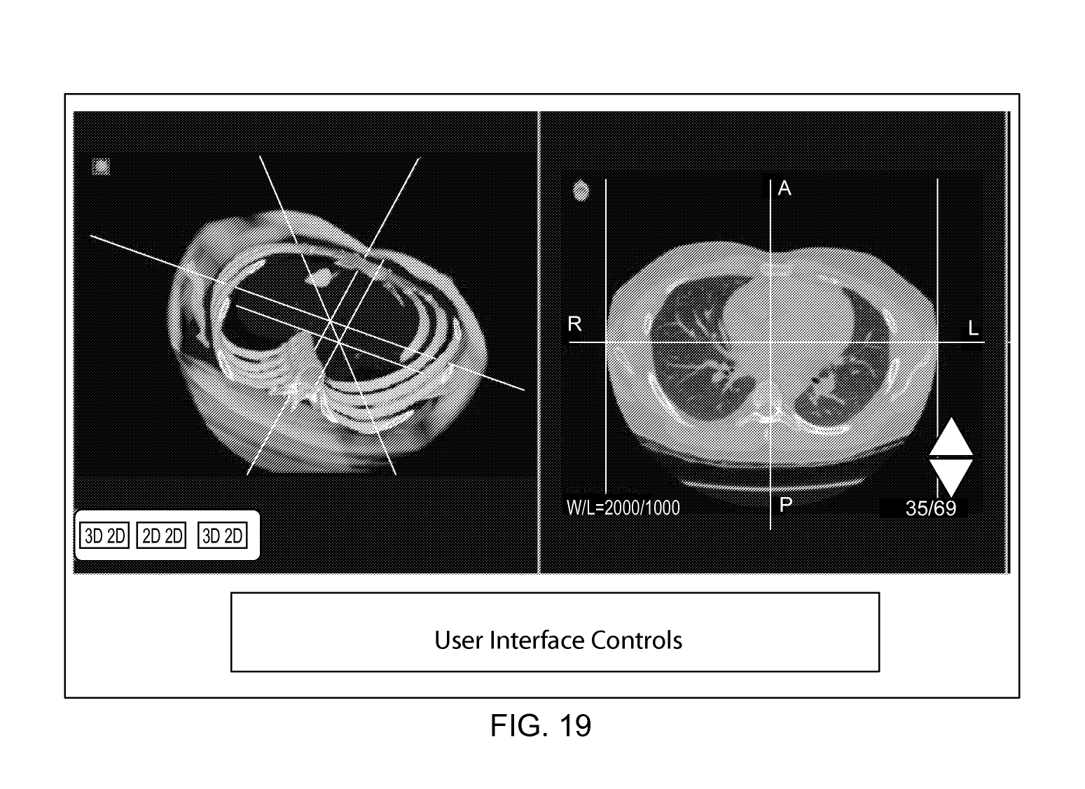

At 562, the image is loaded to a 3D engine. For example, a user can push a “Load Button” available on the user interface. At 564, a 3D volume of the image stack selected is generated and displayed on a 3D window, and a corresponding 2D window can display a selected slice with reference to the 3D volume. FIG. 19 illustrates an example display showing a 3D image and corresponding 2D slice. To create different volumes of interest (VOIs) as may be needed, 3D visualization presets can be selected at 566. At 568, additional VOIs can be created using, for example, cubic and free hand VOI creation tools of the planning station and other segmentation methods available. At 570, the user can identify segmented organs to be avoided by, for example, selecting a “No Go” button. At 572, the user can choose to move to the next step in the planning procedure by, for example, selecting an “Align Probe” button.

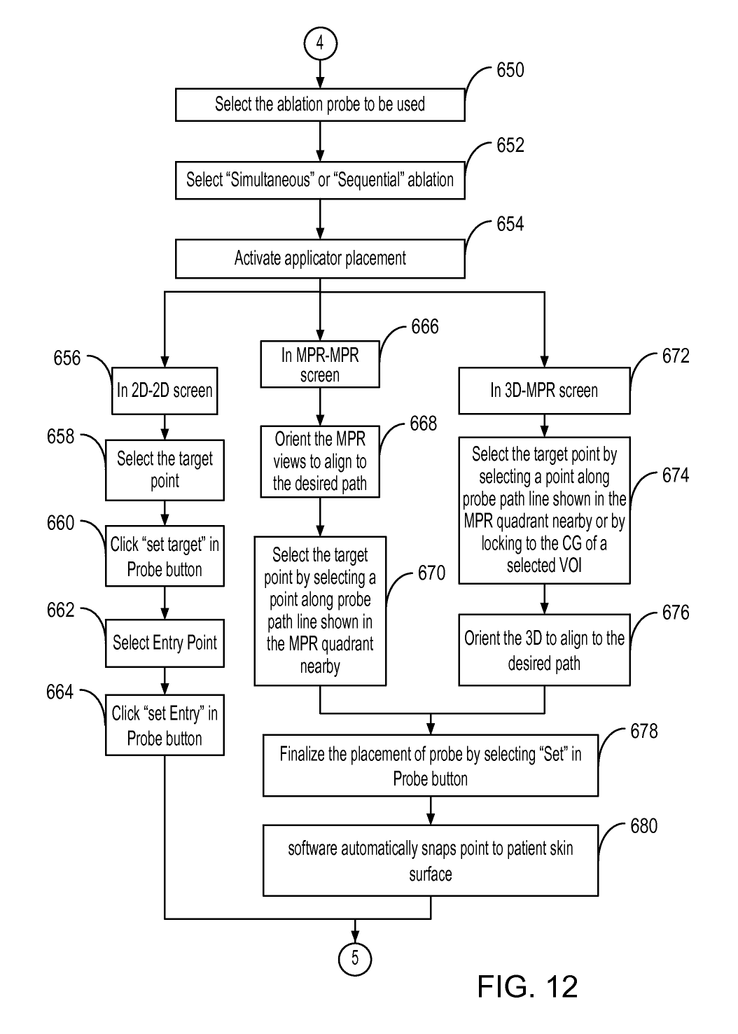

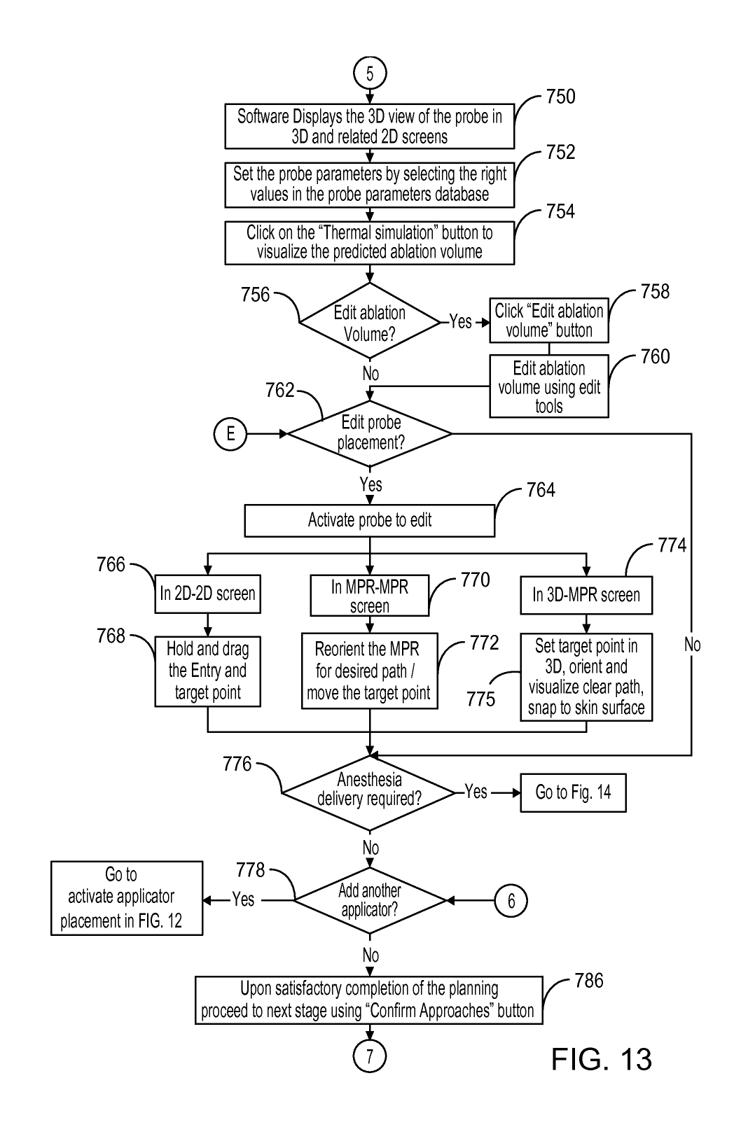

As shown in FIG. 12, at 650, an ablation probe (also referred to herein as “needle” or “ablation needle”) can be selected. At 652 either “simultaneous ablation” or “sequential ablation” can be selected, meaning that the ablation procedure will involve supplying power either simultaneously to multiple ablation probes, or supplying power sequentially to multiple ablation probes or, more typically, to a single ablation probe that is sequentially inserted into two or more positions. At 564 the applicator placement can be activated using one of three options as illustrated in the flow chart of FIG. 12. A first option is to use a 2D-2D screen placement, at 656 in FIG. 12. With this first option, at 658, a target point can be selected. In other words, a selected tissue to be treated can be selected. At 660, the user can select the “Set Target” button. At 662, an entry point for insertion into the patient can be selected, for example, the user can point and click on a selected entry point on the display screen. At 664, a “Set Entry” button can be selected.

A second option is to use a MPR-MPR (multi planar reconstruction) screen placement, as in FIG. 12. At 668, the MPR views can be oriented to align the desired path of insertion into a patient. At 670, the user can select a target point (e.g., tissue with the patient to be treated) by selecting a point along the probe path line shown in the MPR quadrant near the selected target.

A third option is to use a 3D screen placement at 672 in FIG. 12. At 674, the user can select a target point (e.g., tissue within the patient to be treated) by selecting a point along the probe path line shown in the MPR quadrant near the selected target, or by locking to the centroid or center of mass of a selected VOI. At 676, the 3D image is oriented to align to the desired path. If the second or third options are selected, at 678 the user can finalize placement of the probe by, for example, selecting a “Set in Probe” button. At 680, the location on the patient's skin through which the ablation probe will pass to reach the selected placement is determined.

As shown in FIG. 13, a 3D view of the selected probe in a 3D screen and in related 2D screens can be displayed at 750. At 752, the probe parameters can be set. For example, a user can select the correct values from a probe parameters listing stored in the database of the planning station. At 754, a thermal simulation can be activated to visualize the predicted ablation volume. For example, a user can select a “Thermal Simulation” button. At 756, the user can choose to edit the ablation volume. If the user chooses to edit the ablation volume, at 758, the user can select an “Edit Ablation Volume” button, and at 760 the user can edit the ablation volume using editing tools provided. At 762, the user can choose to edit the probe placement. If the user chooses to edit the probe placement, at 764, the user can select the probe to be edited and can select one of three options for viewing the probe placement. A first option is to view in a 2D-2D screen at 766 and hold and drag the entry and target point to move them to a desired location at 768. A second option is to view the probe in a MPR-MPR screen at 770, and reorient the MPR for a desired path and move the target point accordingly at 772. A third option is to view the probe in a 3D-MPR screen at 774. At 775, the user can select the target point in 3D and the target point becomes the center of rotation of the volume. The user can then orient the volume so as to visualize a clear path and select a “set” button. The trajectory is determined based on the orientation of the 3D image and the entry point is automatically selected on the skin surface.

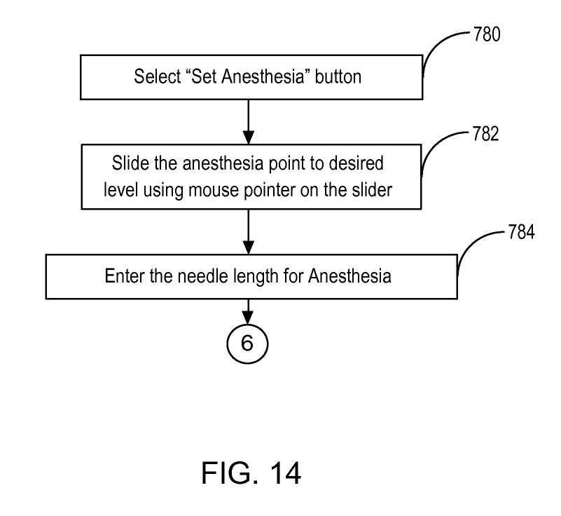

At 776, the user can select if anesthesia delivery is required for the procedure. If anesthesia is required, a “Set Anesthesia” button can be selected at 780, as shown in FIG. 14. An anesthesia point can be displayed along a line between the target point and the entry point and, at 782, the user can move the anesthesia point to a desired location, e.g. by activating a slider on the screen, or other suitable technique. At 784, the user can enter a length for the anesthesia needle. If no anesthesia is needed, at 778, the user can select to add another probe. If no additional probe is needed, the user proceeds to the next stage in the process by selecting a “Confirm Approaches” button at 786. If an additional probe is desired, the user proceeds back to the “activate applicator placement” at 654 in FIG. 12, and repeats the above process for that probe.

The treatment plan generated using the above methods can be stored in a memory of the planning station 200. The treatment plan include, for example, the number of ablation needles to be used, the power levels associated with each probe, the time period for applying power from each probe, the distance between the probes, and the ablation models generated for the probes.

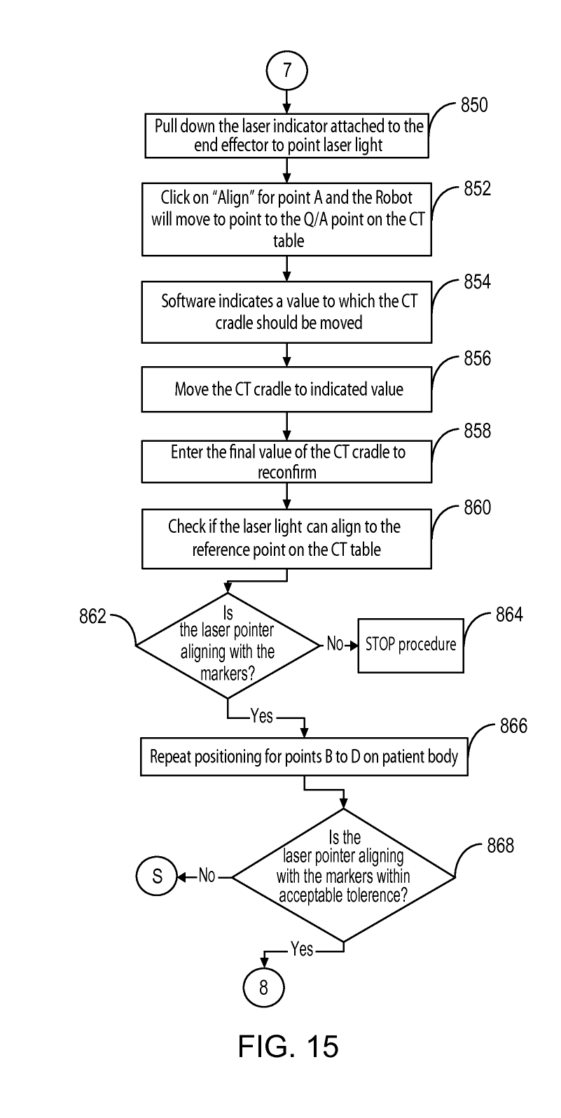

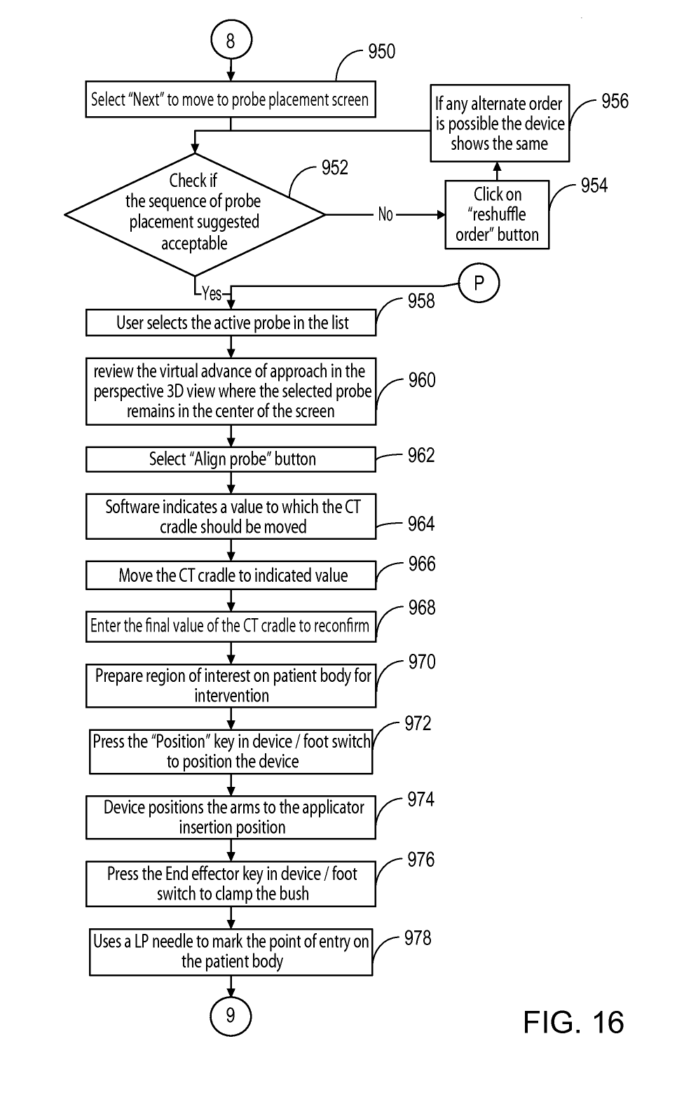

FIGS. 15-18 each illustrate a portion of a method of performing an image-guided interventional procedure, using a treatment plan as generated by the planning station. Referring to FIG. 15, at 850, a laser indicator can be attached to an end effector (also referred to herein as “tool guide”) of the positioning device. At 852, alignment of a reference point(s) on the CT table can be performed. For example, if four reference points are used (referred to here as points A, B, C, and D), the user can select “Align” for point A on the user interface and the positioning device will move to point the laser to a quality assurance (“Q/A”) point on the CT table. The Q/A point is referenced on the CT table to check the proper registration of the device to the CT, when the device is docked (registered to CT). As a safety precaution, just before the procedure is performed, the device is instructed to position the laser light on to the Q/A point so that the user can confirm that the registration is correct. At 854, the software indicates a value to which the CT cradle should be moved. At 856, the CT cradle can be moved to the indicated value. At 858, a final value of the CT cradle can be entered to reconfirm the positioning. At 860, a check can be done to verify that the laser light aligns to the reference point (e.g., point A) on the CT table. At 862, the user can verify if the laser light aligns with the reference markers placed on the patient's body during the set up procedure. If the laser pointer does not align with the markers, the procedure should be stopped. If the laser pointer is aligned with the markers, the positioning can be repeated for points B, C and D on the patient's body at 866. At 868, alignment of the laser pointer to the markers can be checked to be within an acceptable tolerance. If the alignment is not acceptable, the user proceeds to circle S shown in FIG. 10 and repeats the process from that point onward. If the alignment is acceptable, the user can proceed to a probe placement screen at 950, shown in FIG. 16.

As shown in FIG. 16, at 952, the user can check if the sequence of probe placement suggested by the planning station is acceptable. If it is not acceptable, at 954 the order in which the probes are to be inserted can be changed, and at 956, an alternative order of probe placement can be displayed. If the sequence of probe placement is acceptable, at 958, the user can select the active probe from a list stored in the database of the planning station. At 960, the virtual advance of approach can be viewed in a perspective 3D view where the selected probe remains in the center. At 962, the user can actuate alignment of the probe, for example, by selecting an “Align probe” button. At 964, the software of the planning station indicates a value (e.g., location coordinates) to which the CT cradle should be moved. At 966, the CT cradle is moved to the indicated value. At 968, a final value of the CT cradle location is entered to reconfirm. At 970, the region of interest on the patient is prepared for the interventional procedure. At 972 and 974, the positioning device is activated to move to the desired position for the interventional procedure. At 976, an end effector key in the positioning device is activated to clamp a bush member within the end effector. The bush member can be selected according to the gauge of the needle (e.g., ablation tool). At 978, a lumbar puncture (LP) needle can be used to mark the point of entry on the patient body.

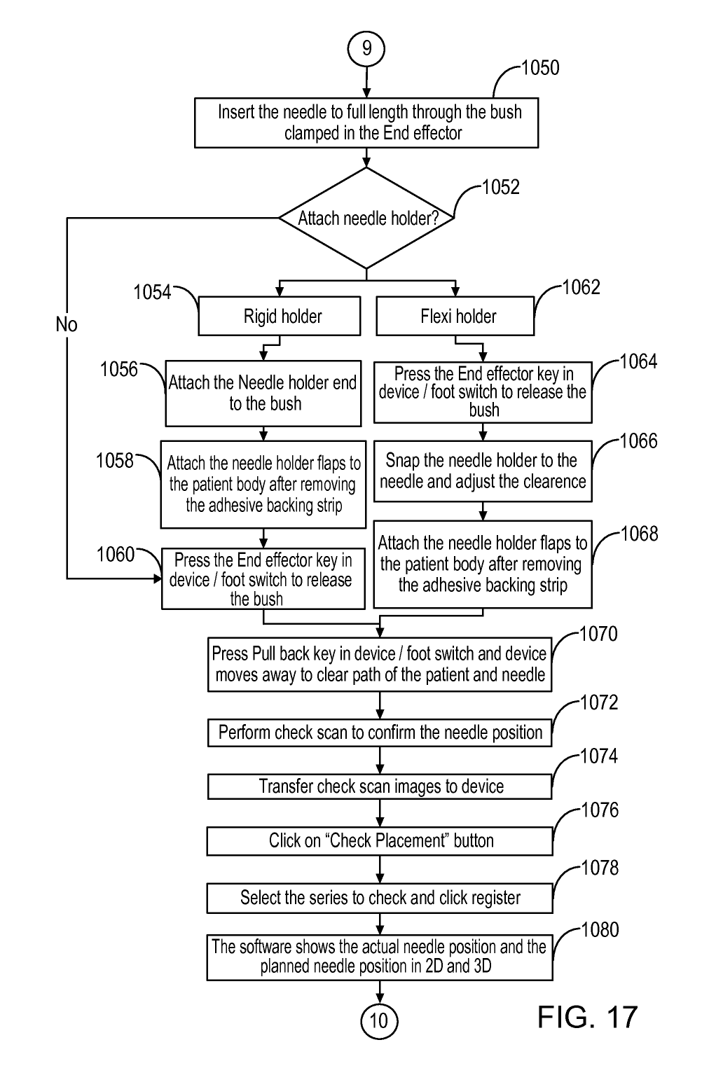

As shown in FIG. 17, at 1050, the ablation needle is inserted to full length through a lumen defined in the bush that is clamped in the end effector. At 1052, a needle holder can optionally be coupled to the patient to provide support for the ablation needle during the interventional procedure. The needle holder can be, for example, a needle holder as described in co-pending U.S. patent application Ser. No. 13/292,186 (the “'186 application”), the disclosure of which is incorporated herein by reference in its entirety. If a needle holder is not to be used for the procedure, the end effector key on the positioning device can be actuated to release the bush (e.g., a foot switch can be actuated) at 1060. If a needle holder is to be used, the user can select between, for example, two different types of needle holder: a rigid needle holder and a flexible needle holder. Examples of such needle holders are described in the ['186] application incorporated by reference above. If a rigid needle holder is selected at 1054, the user can attach an end portion of the needle holder to the bush of the positioning device at 1056. At 1058, the needle holder can be attached to the patient's body. For example, a backing strip on a base portion (e.g., flaps) of the needle holder can be removed and the base portion adhered to the patient's skin. At 1060, the end effector key on the positioning device can be actuated to release the bush form the end effector.

If a flexible needle holder is selected at 1062, the user can press the end effector key to release the bush at 1064. At 1066, the needle holder can be coupled (e.g., snapped) to the ablation needle and the clearance between the needle within an opening in the holder portion of the needle holder can be adjusted. At 1068, the needle holder flaps can be attached to the patient's body (in the same or similar manner as described for the rigid holder at 1058. Next, at 1070, a “Pull Back” key in the positioning device can be pressed (e.g., a foot switch can be actuated) to move the device away from the CT table and to clear a path to the patient and the ablation needle. At 1072, a check scan can be performed to confirm the ablation needle is in the desired position. For example, images of the area of interest and the needle in the patient can be taken. At 1074, the check scan images can be transferred to the positioning device. At 1076, a “Check Placement” button can be actuated to bring up a check placement screen on the display device. At 1078, the user can select the image series to check and select “register” to activate the check placement operation. At 1080, the software can produce on the display device the actual needle position and the planned needle position (from the planning procedure) in 2D and 3D images.

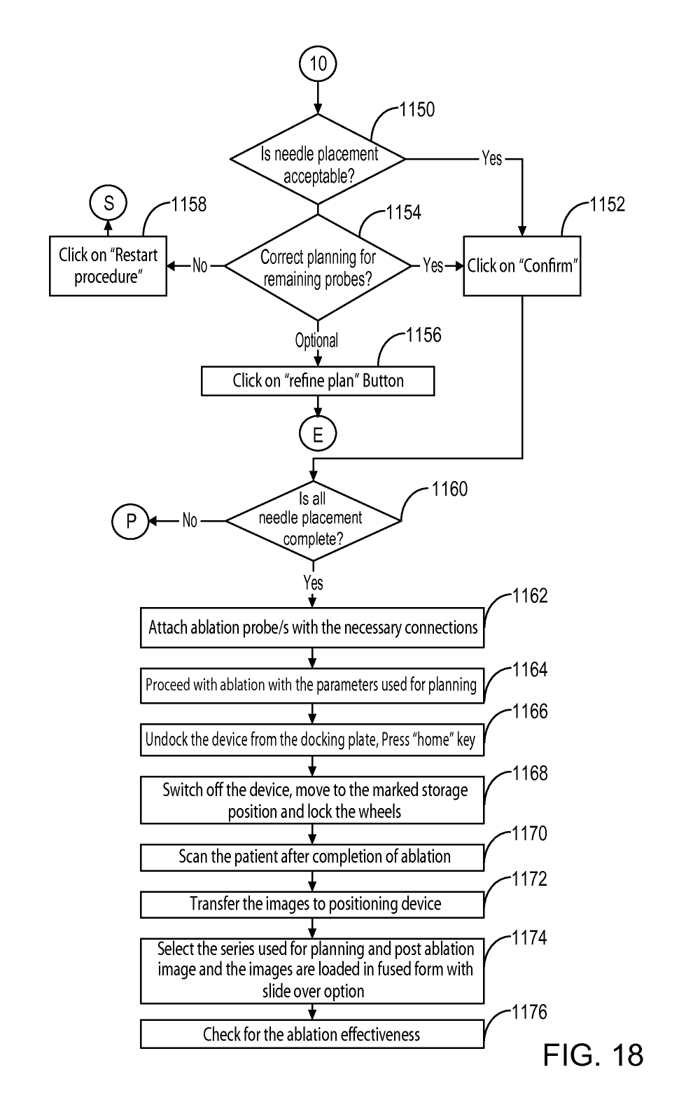

As shown in FIG. 18, a determination can be made as to whether the needle placement is acceptable at 1150. If the needle placement is acceptable, this can be confirmed at 1152. If the needle placement is not acceptable, then at 1154, a determination can be made as to whether or not the planned position of the remaining needle(s) is acceptable in light of the actual placement of the current needle. If the planned position of the remaining needle(s) is acceptable, this can be confirmed at 1152. The plan can optionally be refined at 1156, in which case the user then follows circle E in the flow chart of FIG. 13. If the position of the remaining needles is not acceptable (e.g., at 1154), the procedure can be restarted at 1158, and the user can follow circle S in the flow chart of FIG. 10.

If all the needle placements are complete, at 1060, the ablation needles can be attached to any necessary connections (e.g., power source) at 1062. If the needle placement is not complete at 1160, the user can proceed to circle P in the flow chart of FIG. 16 and repeat the process onward.

At 1064, the ablation procedure can be performed with the parameters used for the planning procedure. When the ablation procedure has been completed, the positioning device can be undocked and a “home” key can be pressed at 1166. The positioning device can be turned off at 1168 and moved to a storage position. Further images can be taken of the area of interest in the patient to determine the effectiveness of the ablation procedure at 1170, and at 1172 the images can be transferred to the positioning device. At 1174, the image series used for planning and the post ablation images can be superimposed and the ablation effectiveness can be checked at 1176.

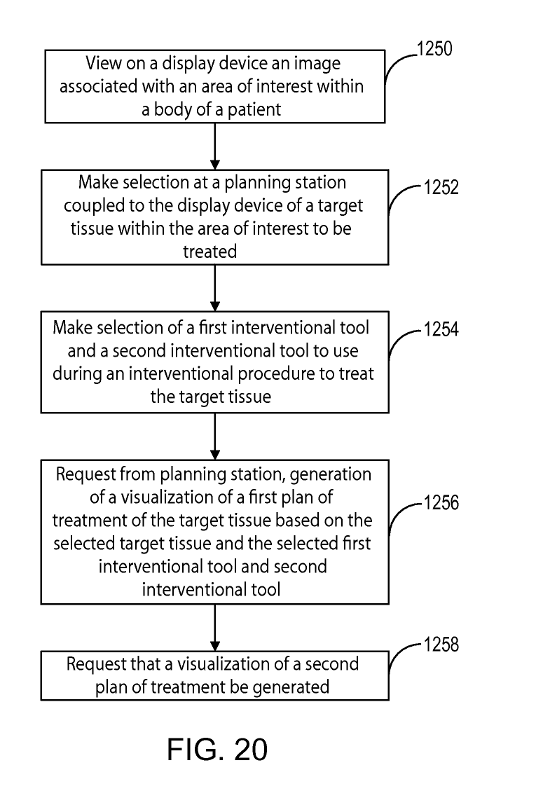

FIG. 20 is a flowchart illustrating a method of creating a plan of treatment, according to an embodiment. The method includes at 1250, viewing on a display device an image associated with an area of interest within a body of a patient. At 1252, a selection of a target tissue within the area of interest to be treated based on the image can be entering at a planning station coupled to and in electrical communication with the display device. At 1254, a selection of a first interventional tool and a selection of a second interventional tool to use during an interventional procedure to treat the target tissue can be entered at the planning station. In some embodiments, the method further includes selecting at the planning station an energy (by selecting a power level and/or a time) to be applied by the first interventional tool and an energy (by selecting a power level and/or a time) to be applied by the second interventional tool.

At 1256, generation of a visualization of a first plan of treatment of the target tissue based on the selected target tissue and the selected first interventional tool and second interventional tool can be requested from the planning station. In some embodiments, a request is made at the planning station for generation of a simulation of the first plan of treatment, and the simulation can be viewable on the display device. During an interventional procedure to treat the target tissue based on the first plan of treatment including insertion of the first interventional tool in the body of the patient, at 1258, a request can be made at the planning station that a visualization of a second plan of treatment be generated. The second plan of treatment can include a change to a selected power level of the second interventional tool, a selected time duration of applying the power level of the second interventional tool, and/or a position of the second interventional tool. In some embodiments, the simulation can be viewable on the display device and can include superimposed images of the first interventional tool, the second interventional tool, and the area of interest within the patient. In some embodiments, the method further includes requesting at the planning station, a simulation of the second plan of treatment, and the simulation of the second plan of treatment can be viewable on the display device.

It is intended that the systems described herein can include, and the methods described herein can be performed by, software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made. For example, although various embodiments have been described as having particular features and/or combinations of components, other embodiments are possible having any combination or sub-combination of any features and/or components from any of the embodiments described herein.

1. A non-transitory processor-readable medium storing code representing instructions to cause a processor to:
receive image data associated with at least one image of an area of interest within a body of a patient, the area of interest including a target tissue to be treated;
display the at least one image on a display device;
receive an input from a user of a selection of a first interventional tool from a plurality of interventional tools about which information is stored in a memory,
receive an input from the user of a selection of a second interventional tool from the plurality of interventional tools about which information is stored in the memory;
execute a simulation viewable on the display device of a treatment plan including disposing the first interventional tool in a first location in the body of the patient, disposing the second interventional tool in a second location in the body of the patient, applying a first amount of energy from the first interventional tool and applying a second amount of energy from the second interventional tool to the body of the patient;
generate a thermal model of the thermal effect collectively produced on the tissue of patient, including the target tissue, by the first interventional tool and the second interventional tool based on the simulation; and
display the thermal model on the display device.

2. The processor readable medium of claim 1, further comprising code to:
receive an input from the user indicating a selected location on the patient to be treated.

3. The processor readable medium of claim 1, further comprising code to:
receive an input from the user of a selection of a power level to be applied during the simulation by the first interventional tool and a selection of a power level to be applied during the simulation by the second interventional tool.

4. The processor readable medium of claim 1, further comprising code to:
receive an input from the user of a selection of a time duration to apply the energy from the first interventional tool during the simulation and a selection of a time duration to apply the energy from the second interventional tool during the simulation.

5. The processor readable medium of claim 1, wherein the simulation viewable on the display device includes superimposed images of the first interventional tool, of the second interventional tool, and of the area of interest within the patient.

6. The processor readable medium of claim 1, wherein the simulation is a first simulation, the processor readable medium further comprising code to:
during an interventional procedure in which a plan of treatment corresponding to the first simulation is being performed, including insertion of the first interventional tool in the body of the patient, receive an input from the user to execute a second simulation different than the first simulation, the second simulation including a change to at least one of a selected power level of the second interventional tool, a selected time duration of applying the power level of the second interventional tool, or a position of the second interventional tool in the body of the patient.

7. The processor readable medium of claim 1, further comprising code to:
during an interventional procedure executing a plan of treatment corresponding to the simulation, receive an image signal associated with at least one image of the area of interest within the body of the patient with the first interventional tool inserted therein.

8. A non-transitory processor-readable medium storing code representing instructions to cause a processor to:
generate a first plan of treatment for an image-guided interventional procedure including treatment of a target tissue within an area of interest within a body of a patient using a first interventional tool and a second interventional tool, the first plan including a predetermined first power level and a predetermined first duration of time applying the first power level for the first interventional tool and a predetermined second power level and a predetermined second duration of time applying the second power level for the second interventional tool;
during an interventional procedure executing the first plan of treatment, receive an image signal associated with at least one image of the area of interest within the body of the patient with the first interventional tool inserted therein; and
receive from a user executing the first plan of treatment, an input indicating a request to generate a second plan of treatment, the second plan of treatment including at least one of a third power level, a third duration of time applying the second or third power level for the second interventional tool.

9. The processor readable medium of claim 8, further comprising code to:
generate a thermal model of the target tissue associated with a thermal effect collectively produced from the first interventional tool and the second interventional tool based on the simulation.

10. The processor readable medium of claim 8, further comprising code to:
prior to the generating a first plan of treatment, receive an input from the user indicating a selected location on the patient to be treated.

11. The processor readable medium of claim 8, further comprising code to:
prior to the generating a first plan of treatment, receive an input from the user of a selection of a power level to be applied during the first plan of treatment by the first interventional tool and a selection of a power level to be applied during the first plan of treatment by the second interventional tool.

12. The processor readable medium of claim 8, further comprising code to:
prior to the generating a first plan of treatment, receive an input from the user of a selection of a duration of time to apply the power from the first interventional tool and a selection of a time duration to apply the power from the second interventional tool.

13. A method, comprising:
viewing on a display device an image associated with an area of interest within a body of a patient;
entering at a planning station, a selection of a target tissue within the area of interest to be treated based on the image;
entering at the planning station, a selection of a first interventional tool and a selection of a second interventional tool to use during an interventional procedure to treat the target tissue;
requesting from the planning station generation of a visualization of a first plan of treatment of the target tissue based on the selecting a target tissue and the selecting a first interventional tool and a second interventional tool; and
during an interventional procedure to treat the target tissue based on the first plan of treatment including insertion of the first interventional tool in the body of the patient, requesting at the planning station that a visualization of a second plan of treatment be generated, the second plan of treatment including a change to at least one of a selected power level of the second interventional tool, a selected time duration of applying the power level of the second interventional tool, or a position of the second interventional tool.

14. The method of claim 13, further comprising:
selecting at the planning station a power level to be applied by the first interventional tool and a power level to be applied by the second interventional tool.

15. The method of claim 14, further comprising:
selecting at the planning station a time duration to apply the power of the first interventional tool and a time duration to apply the power of the second interventional tool.

16. The method of claim 13, further comprising:
requesting at the planning station, a simulation of the first plan of treatment, the simulation being viewable on the display device.

17. The method of claim 16, wherein the simulation viewable on the display device includes superimposed images of the first interventional tool, the second interventional tool, and the area of interest within the patient.

18. The method of claim 13, further comprising:
requesting at the planning station, a simulation of the second plan of treatment, the simulation of the second plan of treatment being viewable on the display device.
